(19) United States
(12) Patent Application Publication
(10) Pub. No.: US 2007/0254264 A1
(43) Pub. Date: Nov. 1, 2007

(54) DENTAL ACCESSORY DEVICE

(76) Inventor: Duc H. NGUYEN, San Diego, CA (US)

Correspondence Address: FRANK G MORKUNAS, 7750 DAGGET ST, SUITE 203, SAN DIEGO, CA, 92111, US

(21) Appl. No.: 11/380,747

(22) Filed: Apr. 28, 2006

(51) Int. Cl. A61C 3/00; A61C 3/04; A61B 19/02

(52) U.S. Cl. 433/163; 433/49; 206/63.5

(57) ABSTRACT

A dental accessory device having a pivot member hingedly attached to a base member and a central opening between the two for receiving a sponge. A latch and corresponding groove or detent hold the pivot member to the base member and one or more teeth-like projections on the inner surface of each member secure the sponge in place. An elongate stem protruding from the bottom of the base member is insertable into an external dental instrument. A dental bur component with hinged cover and a ruler on the top of the device provides easy access to a variety of dental burs and aids in assessing the depth of a root canal being performed, respectively. Attached to the side of the device is a well for holding dental lubricants necessary to the endodontic procedure.

### CROSS REFERENCES TO RELATED APPLICATIONS

None.

### STATEMENT REGARDING FEDERALLY-SPONSORED RESEARCH OR DEVELOPMENT

Not applicable.

### BACKGROUND

This accessory device relates to the profession of dentistry, and more particularly to a dental accessory device mountable onto and removable from a conventional dental instrument wherein the accessory device facilitates immediate access to a dentist of many dental instruments required during routine dental procedures and more in-depth procedures.

In dentistry, the dentist utilizes numerous dental instruments during dental examinations, dental treatments, and dental surgeries. To cope with the need for these many instruments during the various dental procedures, typically a dental assistance is employed to assist the dentist and to provide to the dentist or readily make available to the dentist the particular dental instrument immediately required.

Typically in the endodontic procedures, the dentist must have (1) ready access to rotary files used with a rotary electric motor to negotiate, clean, and shape the root canal into a preferred tapered preparation; (2) ready access to hand files which are small instruments used to manually negotiate, shape, and clean the root canal and, with depth markers, used to assess the depth of the root canal; (3) ready access to a variety of dental burs, small instruments used in a high-speed hand-held dental drill, used to cut the affected tooth and remove decay; (4) ready access to a measuring device [ruler for example] necessary to measure the length of the hand files with or without depth markers so as to measure and assess the depth of a root canal being performed on the patient; and (5) ready access to dental lubricant or dental paste used to lubricate and clean the root canal of debris.

Generally these instruments are grouped together by type and held or contained on a dental tray for easy access by the dental assistant or the dentist. Although access is relatively easy, the detailed work necessary of the root canal render any distraction, such as reaching for and finding the correct instrument, however minimal, and whether done by the dentist or the dental assistance, is nonetheless tedious, cumbersome, and interruptive of the overall procedure.

Moreover, the instruments, though sterile, generally are wiped with a sponge and afterward, if necessary, coated with lubricant prior to use in the procedure. All this activity becomes a waste of precious and, the more instruments which are required of the procedure, the greater the time being wasted. To ameliorate this problem, various concepts have been devised which contain many of the dental instruments needed by the dentist but, though suited for the intended purpose, generally require being worn on the finger of the dentist during the performance of the procedure. This makes availability of the dental instrument virtually immediate but encumbers the dentist by preventing use of all fingers and thereby may jeopardize the dental process.

The novel dental accessory device disclosed in this application cures that potential detriment. It is designed to be mounted on a suitable external object, such as, but not limited to, the distal end of a dental mirror [a dental instrument which is virtually always in the hands of a dentist during the typical endodontic procedure. Moreover, this dental accessory device has an openable elongate socket, with projections on its inner walls which accept and, because of the projections, securely retain a sponge or sponge-like object therein and prevent its accidental removal when removing various instruments held by the sponge.

The foregoing has outlined some of the more pertinent objects of the accessory device. These objects should be construed to be merely illustrative of some of the more prominent features and applications of the accessory device. Many other beneficial results can be attained by applying the disclosed accessory device in a different manner or by modifying the accessory device within the scope of the disclosure. Accordingly, other objects and a fuller understanding of the accessory device may be had by referring to the summary of the accessory device and the detailed description of the preferred embodiment in addition to the scope of the accessory device defined by the claims taken in conjunction with the accompanying drawings.

### SUMMARY

The above-noted problems, among others, are overcome by the accessory device. Briefly stated, the accessory device contemplates a dental device with a pivot member hingedly attached to a base member and a central opening between the two members for securely containing a sponge-like member. A latch and corresponding groove or detent hold the pivot member to the base member with the sponge-like member in place. One or more teeth-like projections on the base member and the pivot member secure the sponge-like member inside the opening.

An elongate stem protrudes from the bottom of the base member and is adapted to insert into an external dental instrument. The elongate stem has five or more elongate sides for a secure fit into the external dental instrument and for a position best suited to the needs or preferences of the user. On the top of the device is a measuring indicia to aid the user in assessing the depth of the root canal being performed. Attached to the side of the device also is a well for containing dental lubricants or pastes necessary to the user for the endodontic procedure.

The foregoing has outlined the more pertinent and important features of the accessory device in order that the detailed description that follows may be better understood so the present contributions to the art may be more fully appreciated. Additional features of the accessory device will be described hereinafter which form the subject of the claims. It should be appreciated by those skilled in the art that the conception and the disclosed specific embodiment may be readily utilized as a basis for modifying or designing other structures and methods for carrying out the same purposes of the accessory device. It also should be realized by those skilled in the art that such equivalent constructions and methods do not depart from the spirit and scope of the accessory device as set forth in the appended claims.

### BRIEF DESCRIPTION OF THE DRAWINGS

For a fuller understanding of the nature and objects of the accessory device, reference should be had to the following detailed description taken in conjunction with the accompanying drawings in which:

FIG. 1 is a perspective view of the dental accessary device.

FIG. 2 is a top plan view of the dental accessary device in a closed position.

FIG. 3 as taken on line 3-3 of FIG. 2 is detailed elevation view of the pivotal member of the dental accessary device.

FIG. 4 as taken on line 4-4 of FIG. 2 is detailed elevation view of the base member of the dental accessary device.

FIG. 5 is a top plan view of the dental accessary device in an open position.

FIG. 6 is a front elevation view of the dental accessary device containing various dental instruments.

FIG. 7 as taken on line 7-7 of FIG. 6 is a side elevation view of the dental accessary device.

### DETAILED DESCRIPTION

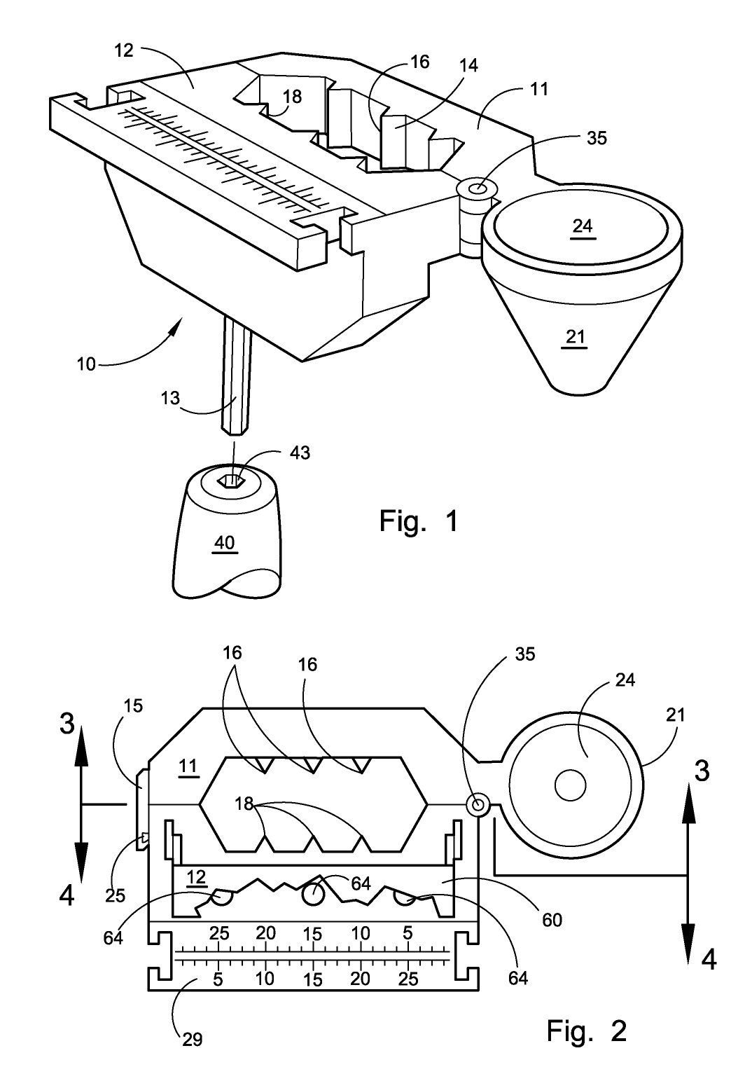

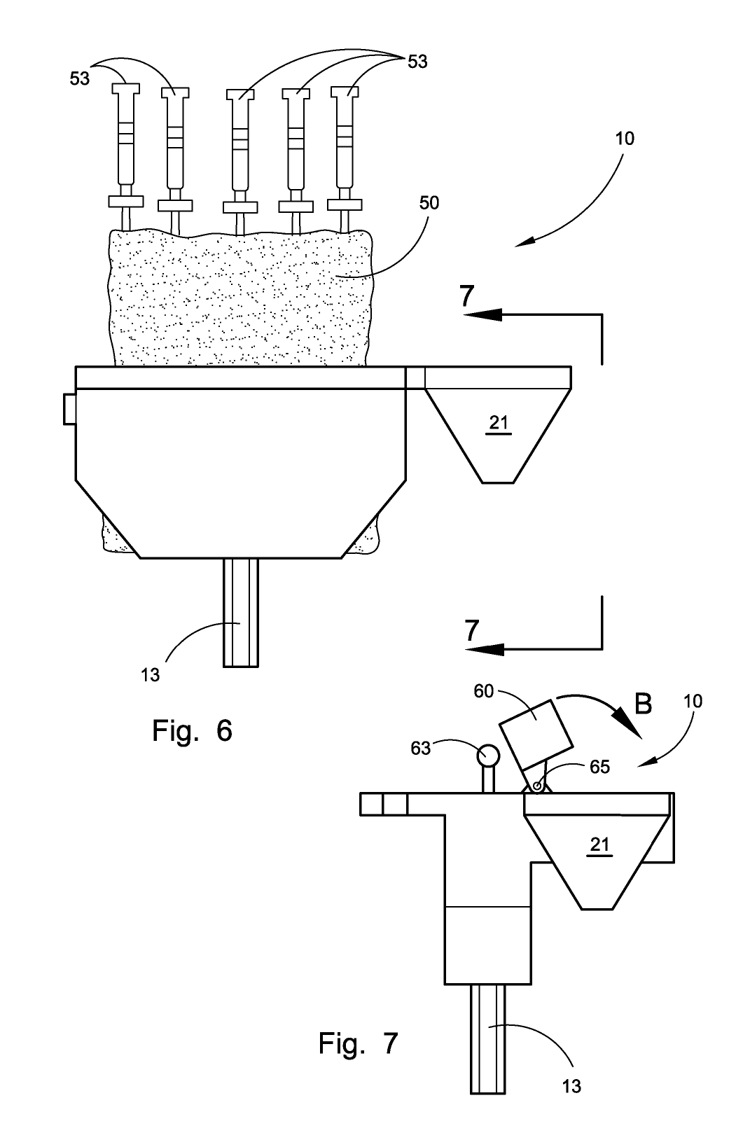

Referring now to the drawings in detail and in particular to FIG. 1, reference character 10 generally designates a dental accessory device constructed in accordance with a preferred embodiment. The device 10 has a base 12 and a pivot member 11 attached to the base 12 by a hinge 35 permitting the pivot member 11 to be swung open in the direction of Arrow A of FIG. 5. A latch 15 on the pivot member 11 and corresponding catch, detent 25 or groove 25′ on the base 12 [as illustrated in FIGS. 2 and 5] securely maintains the pivot member 11 to the base 12 in a closed position.

An elongate stem 13 extends from the bottom of the base 12 and is insertable into an external dental implement 40 such as, but not limited to, the handle of a dental mirror wherein the handle has a corresponding receptacle 43 at its top. The stem 13 has five or more elongate sides; i.e., pentagonal, hexagonal, octagonal, and the like. This permits a fitting of the device 10 into the external implement 40 best suited to the needs or preferences of the user. The stem 13 may be made of the same material and from the same mold as is the device 10 or preferably, it may be made of a different and metallic material.

An elongate opening 14 is defined centrally between the space formed when the device 10 is in the closed position. The function of this opening 14 is to house and securely maintain an external object 50 which is suited to contain one or more dental tools required of the user and is further suited to provide the means for the user to wipe the dental tools when and if needed. The external object 50 suited for these intended purposes include, but are not limited to, sponges, foam rubber, polyethylene foam materials, polystyrene plastics, and any other elastic porous mass.

FIG. 6 illustrates the device 10 with a sponge 50 held in the elongate opening 14. Contained atop the sponge 50 are several dental tools 53 such as, but not limited to, one or more dental rotary files, one or more dental hand files, and one or more dental burs, for extremely ready access to the user. As illustrated in this figure and FIG. 6, as well as several other figures, a well 21 is at the far end of the device 10. As shown in the figures, the well 21 is an integral part of the pivot member 11 but it must be understood that this is not a limitation to this device 10. As such the well may instead be an integral part of the base 12.

The well 21 also has a well chamber 24 which is adapted to contain a lubricant or paste necessary to the user during the endodontic procedure. All items [rotary files, hand files, burs, and lubricant/paste] are within immediate access to the user.

In using the various files or burs, the user will pull out which one needed, tend to the procedure, and replace it when done. This process may be repeated several times during the endodontic procedure until the user is satisfied with the final result. Such repeated pulling outward will generally cause the sponge 50, unless it is glued in place, to become less secure in the opening 14 or even pull out from the opening 14. If glued in place, when the sponge 50 wears out from repeated uses, the device 10 would generally be discarded.

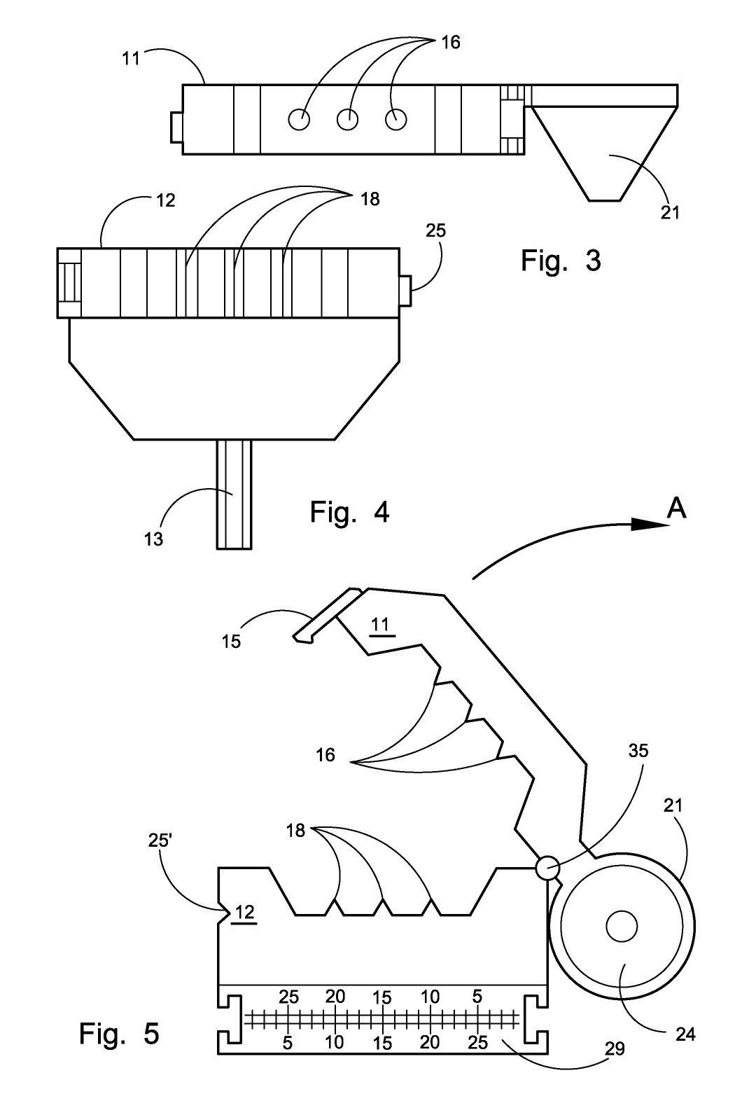

In this novel device 10, however, the sponge 50 is securely held in place by a one or more teeth-like projections 16 on the pivot member 11 and one or more teeth-like projections 18 on the base 18. As illustrated in FIGS. 3 and 4 these projections 16, 18 may be vertically disposed on the entire inner surface of either the base 12 [FIG. 4 refers] or the pivot member 11, or these projections 16, 18 may be cone-shaped [FIG. 3 refers].

In any event, with the device 10 in the open position [FIG. 5], a sponge-like material 50 is inserted into the open space of either the base 12 or the pivot member 11 and the device is placed into the closed position [FIGS. 1, 2, and 6]. The projections 16, 18 bite into the sponge-like member 50 and secure it therein. By so doing it will prevent the sponge-like member 50 from translating down or up and out of the opening 14 as various dental tools are being removed from the sponge-like member 50 and later being replaced.

A final feature of this device 10 includes measuring indicia 29 on the top of the base 12. This is important to the user in measuring the depth of the root canal as the process evolves. The user typically will use one of the hand files, for example, with a stopper on the blade-side of the file and place the file down canal to its end. The stopper then is moved down the file to the top of the tooth. The stopper positioned there and the file is removed from the canal with stopper in place. The distance is then measured using the measuring indicia 29 of the dental accessory device 10. When a different file or rotary file is later used in the procedure, the user will place a stopper on the instrument commensurate with the depth of the canal so that no damage be done to the patient's nerve endings.

In another embodiment, on the base top 12 is a plurality of apertures 64 which are designed to contain one or more dental burs 63 [FIGS. 2 and 7 refer]. A cover 60 encases the dental burs 63 thereby protecting them until needed. The cover 60 is hingedly attached to the base 12 with hinges 65 on each side thereby permitting the cover 60 to be opened as needed [as illustrated by directional Arrow B in FIG. 7].

The present disclosure includes that contained in the present claims as well as that of the foregoing description. Although this accessory device has been described in its preferred forms with a certain degree of particularity, it is understood that the present disclosure of the preferred form has been made only by way of example and numerous changes in the details of construction and combination and arrangement of parts may be resorted to without departing from the spirit and scope of the accessory device. Accordingly, the scope of the accessory device should be determined not by the embodiments illustrated, but by the appended claims and their legal equivalents.

Applicant has attempted to disclose all the embodiments of the accessory device that could be reasonably foreseen. It must be understood, however, that there may be unforeseeable insubstantial modifications to accessory device that remain as equivalents and thereby falling within the scope of the accessory device.

What is claimed is:

1. A dental accessory device comprising:
(a) a base member having a bottom, a top, and an inner surface;
(b) a pivot member having an inner surface and hingedly attached to said base member at a first side of said device wherein an elongate opening is defined between said base member inner surface and said pivot member inner surface and wherein said elongate opening is adapted to receive an external object; and
(c) one or more projections on said base member inner surface and one or more projections on said pivot member inner surface, said projections adapted to retain said external object in said elongate opening.

2. The device of claim 1 wherein said one or more projections are vertically disposed on said pivot member inner surface and on said inner surface base member.

3. The device of claim 1 wherein said one or more projections are cone-shaped projections.

4. The device of claim 1 further comprising latching means on a second side of said device for securing said pivot member to said base member when said pivot member is pivoted to said base member.

5. The device of claim 1 further comprising a well attached to said first side of said device.

6. The device of claim 1 further comprising measuring indicia on the top of said base member.

7. The device of claim 1 further comprising an elongate stem protruding from the bottom of said base member wherein said elongate stem is adapted to insert into an external dental implement having a corresponding receptacle.

8. The device of claim 7 wherein said elongate stem comprises five or more elongate sides.

9. The device of claim 1 wherein said external object is selected from a group consisting of sponges, foam rubber, any elastic porous mass, polyethylene foam materials, and polystyrene plastics.

10. The device of claim 9 wherein said external object is adapted to receive and retain dental drills, dental files, and dental burs.

11. The device of claim 1 further comprising one or more apertures on the top of said base member wherein said one or more apertures is adapted to receive dental burs.

12. The device of claim 11 further comprising a bur cover hingedly attached to the top of said base member.

13. A dental accessory device comprising:
(a) a base member having a bottom, a top, and an inner surface;
(b) a pivot member having an inner surface and hingedly attached to said base member at a first side of said device wherein an elongate opening is defined between said base member inner surface and said pivot member inner surface and wherein said elongate opening is adapted to receive an external object;
(c) one or more projections on said base member inner surface and one or more projections on said pivot member inner surface, said projections adapted to retain said external object in said elongate opening; and
(d) latching means on a second side of said device for securing said pivot member to said base member when said pivot member is pivoted to said base member.

14. The device of claim 13 wherein said one or more projections are vertically disposed on said pivot member inner surface and on said inner surface base member.

15. The device of claim 13 wherein said one or more projections are cone-shaped projections.

16. The device of claim 13 further comprising a well attached to said first side of said device.

17. The device of claim 13 further comprising measuring indicia on the top of said base member.

18. The device of claim 13 further comprising an elongate stem protruding from the bottom of said base member wherein said elongate stem is adapted to insert into an external dental implement having a corresponding receptacle.

19. The device of claim 18 wherein said elongate stem comprises five or more elongate sides.

20. The device of claim 13 wherein said external object is selected from a group consisting of sponges, foam rubber, any elastic porous mass, polyethylene foam materials, and polystyrene plastics.

21. The device of claim 20 wherein said external object is adapted to receive and retain dental drills, dental files, and dental burs.

22. The device of claim 13 further comprising one or more apertures on the top of said base member wherein said one or more apertures is adapted to receive dental burs.

23. The device of claim 22 further comprising a bur cover hingedly attached to the top of said base member.
